(19) United States
(12) Patent Application Publication
(10) Pub. No.: US 2004/0220613 A1
(43) Pub. Date: Nov. 4, 2004

(54) Surgical instrument

(76) Inventor: Peter Petros, South Perth (AU)

Correspondence Address: Lisa J. Moyles, U.S. Surgical, a division of TYCO, Healthcare Group, LP, 150 Glover Avenue, Norwalk, CT, 06856, US

(21) Appl. No.: 10/858,736

(22) Filed: Jun. 2, 2004

(30) Foreign Application Priority Data
Dec. 20, 2001 (AU) PR 9697
Dec. 20, 2002 (WO) PCT/IB02/05783

(51) Int. Cl. A61B 17/06

(52) U.S. Cl. 606/222; 606/223

(57) ABSTRACT

A surgical instrument (10) intended for use in location of a filament in a body, where the instrument comprises a rigid shaft (12) having a handle (14) at one end (16), the other end (18) of the shaft being convergent and formed with an eye (22) adjacent to the other end, the eye being dimensioned to receive the filament, the shaft being curved in a portion proximate the other end.

### BACKGROUND

1. Technical Field

This invention relates to a surgical instrument which can be utilized for the insertion of a filament into the body, and more particularly, to a surgical instrument for use in placement of a support in the body to relieve vaginal prolapse.

2. Background of Related Art

Normal vaginal delivery exposes the female pelvic floor to muscle and connective tissue trauma which, in some cases, can result in pelvic floor retraction and pelvic organ prolapse. Vaginal prolapse is a result of weakening of connective tissue support to the vaginal vault apex. One of the most common surgical techniques used to correct vaginal prolapse includes tying the upper part of the vagina to a connective tissue condensation stretched from both sides of the sacrum. Placement and securement of a support in this area of the body is often difficult.

### SUMMARY

Accordingly the disclosure relates to a surgical instrument intended for use in location of a filament in a body, the instrument includes a rigid shaft having a handle at one or a first end, the other or second end of the shaft being convergent and formed with an eye adjacent to the second end, the eye being dimensioned to receive the filament, the shaft being curved in a portion proximate the second end.

According to on embodiment the eye is formed with a slot which opens laterally to one side of the shaft. According to a preferred feature of the invention the eye has an axial dimension which is greater than the transverse dimension. According to a preferred feature of one embodiment, the slot has a transverse dimension which is less than the axial dimension.

According to a preferred feature of the instrument the portion proximate the second end comprises approximately one quarter to one half of the axial extent of the shaft.

According to a preferred feature of the instrument the handle includes a pair of handle members which extend transversely from the one end of the shaft in opposed relation to each other. According to a preferred feature of the instrument the handle members have a generally triangular shape. According to a preferred feature of the instrument the plane which includes the handle members is generally perpendicular to the portion of the shaft proximate the second end.

The disclosed surgical instrument will be more fully understood in the light of the following description of one specific embodiment.

### BRIEF DESCRIPTION OF THE DRAWINGS

The description is made with reference to the accompanying drawings of which:

FIG. 1 is an isometric view of a surgical instrument according to the embodiment; and

FIG. 2 is an enlarged view of the other end of the shaft incorporating the eye.

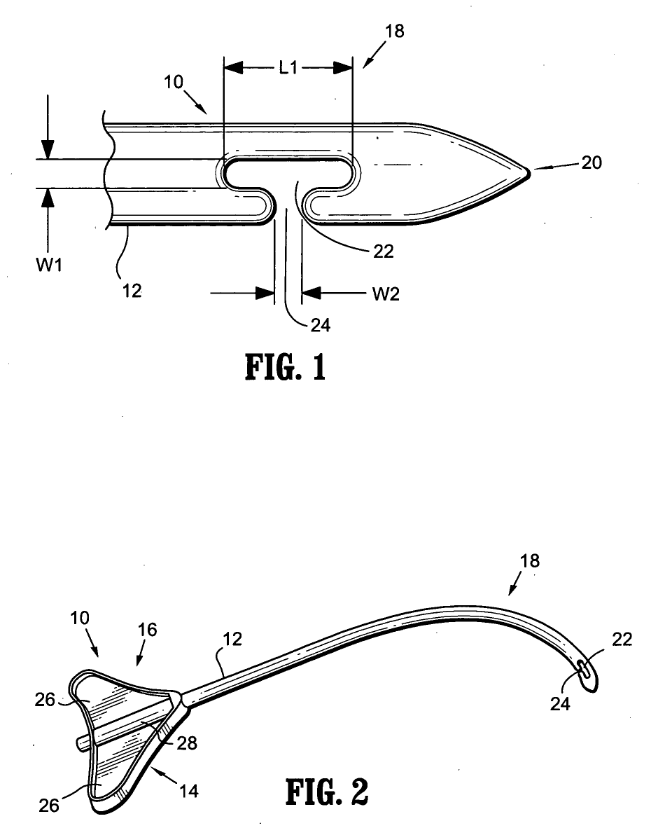

### DETAILED DESCRIPTION OF PREFERRED EMBODIMENTS

The embodiment is directed to a surgical instrument 10 which can be utilised for the location of a filament comprising a tape formed of a suitable material into the body. An application of the embodiment relates to the insertion of a tape from the tissue surrounding the vagina into the vaginal cavity for the purposes of supplementing or replacing support for the vagina in the event of vaginal prolapse.

Surgical instrument 10 includes a shaft 12 having a handle 14 at a first end16. A second end 18 of the shaft 12 is convergent to provide a substantially pointed end 20. The outer most third (approximately) of the shaft 12 is curved through substantially 90 degrees. The second end 18 is provided with an eye 22 adjacent to the second end 18 where the eye 22 has an axial dimension L1 which is greater than the transverse dimension W1. In addition the eye 22 is provided with a slot 24 which extends transversely from the eye 22 to the outer wall of the shaft 12. The width W2 of the slot 24 is less than the axial dimension L1 of the eye 22 and as a result the eye 22 and slot 24 jointly define an opening which extends transversely across the shaft 12 which has a generally keyhole like cross-sectional configuration.

The handle 14 comprises a pair of lateral portions or “wings” 26 which extend to either side of a central tubular boss 28 which receives the shaft 12. The plane of the handle 14 is generally perpendicular to the plane of the curved shaft 12.

The surgical instrument 10 according to the embodiment provides a means whereby a length of tape can be accurately located in position in the patient's body when used in relation to an operation involving resolution of vaginal prolapse.

In use the shaft 12 is inserted into the body through an incision and is caused to pass through the body and to penetrate the vaginal wall through a preformed incision. Once in that location the eye 22 can be accessed through the vagina to allow insertion of a length of tape through the eye 22. With the withdrawal of the shaft 22 from the body, the tape can then be drawn through the body to extend from the vagina to the exterior of the body. This process is then repeated to the opposite side of the body to provide access between the eye 22 of the shaft and the remaining length of tape received within the vagina to enable that portion of the tape to be extended through the body. The ends of the tape may then be fixed to tissue within the body in order to supplement or replace pre-existing support.

The curvature of the shaft 12 facilitates manipulation and location of the eye 22 while the configuration of the eye 22 readily enables the tape to be inserted in position in the eye 22 and without undue dexterity being required.

Throughout the specification, unless the context requires otherwise, the word “comprise” or variations such as “comprises” or “comprising”, will be understood to imply the inclusion of a stated integer or group of integers but not the exclusion of any other integer or group of integers.

It should be appreciated that the scope of the present disclosure need not be limited to the particular scope of the embodiment described. For example other configurations of the eye and slot may be provided to use with other types of supports. Additionally, the eye may be located in other locations adjacent the second end of the shaft.

What is claimed is:

1. A surgical instrument intended for use in location of a filament in the body of a patient, said instrument comprising a rigid shaft having a handle at a first end, a second end of the shaft being formed with an eye adjacent to the osecond end, said eye being dimensioned to receive said filament, the shaft being curved in a portion proximate the other end.

2. A surgical instrument as claimed in claim 1 wherein the shaft is convergent.

3. A surgical instrument as claimed in claim 1 wherein the eye is formed with a slot which opens laterally to one side of the shaft.

4. A surgical instrument as claimed in claim 1 wherein the eye has an axial dimension which is greater than the transverse dimension.

5. A surgical instrument as claimed in claim 4 wherein the slot has a transverse dimension which is less than the axial dimension of the eye

6. A surgical instrument as claimed in claim 1 wherein the portion proximate the second end comprises approximately one quarter to one half of the axial extent of the shaft.

7. A surgical instrument as claimed in claim 1 wherein the handle comprises a pair of handle members which extend transversely from the first end of the shaft in opposed relation to each other.

8. A surgical instrument as claimed at claim 7 wherein the handle members have a generally triangular shape.

9. A surgical instrument as claimed in claim 7 wherein the plane which includes the handle members is generally perpendicular to the portion of the shaft proximate the second end.

10. A method of inserting a support in a body comprising:
providing a surgical instrument having a shaft and an eye in the shaft at a distal end;
positioning the shaft through a first side the body such that the eye of the shaft is positioned within the virginal wall;
engaging a support with the eye of the shaft;
withdrawing the shaft to pull a portion of the support outside the body; and
repeating the procedure through a second side of the body.
